(19) United States
(12) Patent Application Publication
(10) Pub. No.: US 2018/0165757 A1
(43) Pub. Date: Jun. 14, 2018

(54) Purchase health care system

(71) Applicant: Arthur Morris Gelber, Ridgefield, CT (US)

(72) Inventor: Arthur Morris Gelber, Ridgefield, CT (US)

(21) Appl. No.: 15/731,087

(22) Filed: Apr. 17, 2017

Related U.S. Application Data
(60) Provisional application No. 62/323,409, filed on Apr. 15, 2016.

(51) Int. Cl. G06Q 30/08; G16H 10/60

(52) U.S. Cl. CPC G06Q 30/08; G16H 10/60

(57) ABSTRACT

An electronic automated purchase care exchange network system for purchasing healthcare from the healthcare industry and as an intermediary, selling healthcare directly to the public. The system processes free and ubiquitous healthcare and insurance industry metadata to emulate industry protocols, learning from a set examples, applying training dataset to learning algorithms, applying ‘test’ set to assess the performance of fully-specified classifiers, for ensuring compliance during financial transactions. Accordingly, non-compliant bids are transformed into compliant proffers. Unlike health insurance coverage, the intermediary pays for the delivery of healthcare services, accepts financial risk for the delivery of healthcare services and establishes, operates or maintains an arrangement or contract with healthcare providers relating to (a) the healthcare services rendered by the providers, and (B) the amounts to be paid to the providers for such services. The application eliminates medical and insurance administration of claims while mitigating the risks of fraud and abuse.

The present invention relates to a method of selling health care services, providing unrestricted access to healthcare providers and facilities, to consumers searching via the Internet for health care services and the buying of healthcare related services from healthcare providers, facilities and suppliers. These are commercial transactions conducted on the Internet between a device and web site.

### TECHNICAL FIELD

This invention relates to systems and methods for selling healthcare related services to consumers and buying health care services from providers, and establishing methods for introducing pricing compliance prior to purchasing a healthcare services.

### BACKGROUND OF THE INVENTION

Healthcare pricing compliance issues represent up to 10% of healthcare expenditures. In 2015, U.S. healthcare increased 5.8% to reach $3.2 trillion, or $9,990 per person. The 5.8% is the average project growth rate over the next 5 years. In 2016, healthcare pricing compliance issues cost per person, exceeds $1,000 annually. In addition to pricing compliance issues payments cost sharing arrangements between the benefit plan and the insured continue to increase the financial liability onto the consumer. The Kaiser Family Foundation analysis reported in 2016, “that for workers covered by their employer's health plans, out-of-pocket costs including deductibles and coinsurance have be increasing significantly faster than costs paid by insurers.” These financial barriers are having a significant impact on access to care. The present invention monitors-audits, ensuring pricing compliance, whereby delivering unrestricted access to healthcare services that are affordable for those in need of care.

Medicare's research identifiable files, public uses files (PUF) data files are downloadable and were first made available in CY1999. The Centers for Medicare & Medicaid Services (CMS) has developed this data to enable researches and policymakers to evaluate geographic variation in the utilization and quality of health care services, including charges and reimbursement rates healthcare providers, facilities, suppliers, etc. The information provides information on services and procedures; utilization and payment data. Additionally, since 1996 the Medicare National Correct Coding Initiative (NCCI) procedure to procedure (PTP) edits have been implemented to promote NCCI methodologies to control improper billing leading to inappropriate payment.

US Application US20110022479A1, Henley, publication date 2011 Jan. 27 Method and system for providing an on-line healthcare open market exchange and US Application US20050182660A1, Henley, publication date 2005 Aug. 18 and US Application, Business method and system for providing an on-line healthcare market exchange and US20020069085A1 to Engel and Heisen, publication date 2002 Jun. 6, System and method for purchasing health-related services do not address improper payments in their respective schemata. On the issue of pricing compliance, prior art methodology is negligible.

Today, pricing compliance is a $350 billion-dollar issue, projected to grow at a 5.8% rate annually. This represents a failure to meet a certain standard in their systems and methodologies, which is leading to inappropriate purchases. Within the context of purchasing health-care related services, albeit an online auction, prior art lacks the sufficient knowledge or processes to confront the issue of compliance and cost-sharing arrangements. Consumers cannot afford to make inappropriate payments for health-care related services. Healthcare providers are required to meet compliance standards to prevent improper payments. In this context, prior art has placed the buyer and the seller at unnecessary risk. The present invention extracts the Medicare Public Use Files, including proprietary databases, to introduce pricing compliance and cost-savings methodologies automatically, prior to buying or selling health-care services. The present invention is at the forefront of purchasing health-care related services.

### SUMMARY OF THE INVENTION

The present invention has been made in view of the above circumstances and has an objective to provide an improved healthcare delivery system that responds to escalating medical billing and insurance administrative costs via a purchase care exchange system.

A further objective of the invention is to provide an automated electronic purchase care exchange system that provides unrestricted access to affordable healthcare with transparency of price and quality of care data.

A further object of the invention is to provide an automated electronic purchase care exchange system that ensures accuracy and compliance with conventional protocols.

A further object of the invention is to provide an automated electronic purchase care exchange system that performs purchasing operations without overpaying for healthcare due to a lack of compliance and/or fraud and abuse.

A further object of the invention is to provide an automated electronic purchase care exchange system that may be remotely controlled.

A further object of the invention is to provide automated funding solutions associated with the execution of a transactions related to purchasing opportunities.

### BRIEF DESCRIPTION OF THE DRAWINGS

The accompanying drawings, which are incorporated in and constitute a part of this specification, illustrates embodiment(s) of the invention and together with the description, serve to explain the principles of the invention.

FIG. 1 illustrates an embodiment of an automated electronic purchase care exchange system network in accordance with the present invention.

FIG. 1a illustrates a further embodiment of an automated electronic purchase care exchange system in accordance with the present invention.

FIGS. 1a.1 1b, 1c, 1d collectively referred to in the description of the preferred embodiment as FIG. 1, illustrate an embodiment of an automated electronic purchase care exchange system for use in connection with an actor in accordance with the present invention, with 1a being an illustration a remote user accessing a first computer, with 1a.1 being an illustration the inventory component updating inventory market feed with supplier registrant purchase orders for healthcare, with 1 b being an illustration of building models from source data,

FIG. 2 being an illustration of transactional details flowing between the a user's intelligent device and the present invention

FIG. 2a illustrates a further embodiment of an automated electronic purchase care exchange system network in accordance with the present invention.

FIG. 3 provides a schematic of the of the functionality of an embodiment of an automated electronic purchase care exchange system network in accordance with the present invention.

FIG. 4 provides illustrates the transformation from a bid to a compliant proffer

FIG. 5, 5a, 5b, provides a flow diagram of the steps performed in purchasing healthcare from suppliers of healthcare and buyers of healthcare in accordance with the present invention.

FIG. 6, 6a, illustrates steps buyer steps to purchase care.

FIG. 7, 7a, 7b illustrates supplier registration

### DESCRIPTION OF THE PREFERRED EMBODIMENT

The present invention recognized that healthcare coverage continues to diminish while administrating medical billing and insurance claims costs are rising. Prospectively, these costs will continue to negatively impact access to affordable quality of care. Accordingly, introducing business and clinical intelligence during the purchasing of care would curb rising healthcare cost while increasing competition amongst stakeholders in a patient centric-data driven marketplace.

The present invention is capable of reducing administrative costs and broadening access to affordable quality healthcare without the hassles traditionally associated with the current healthcare delivery system. In accordance with one aspect of the present invention, the automated purchase care exchange system enables the public to engage in comparative shopping online for a healthcare provider, ‘name your own price’, select your healthcare provider, and schedule your appointment without insurance. The system applies pre-purchase edits to counter a bid with a compliant proffer. The system will eliminate any excessive charges, unnecessary healthcare, or hidden costs from the bid making a counter-compliant proffer minus the adjustments affordable.

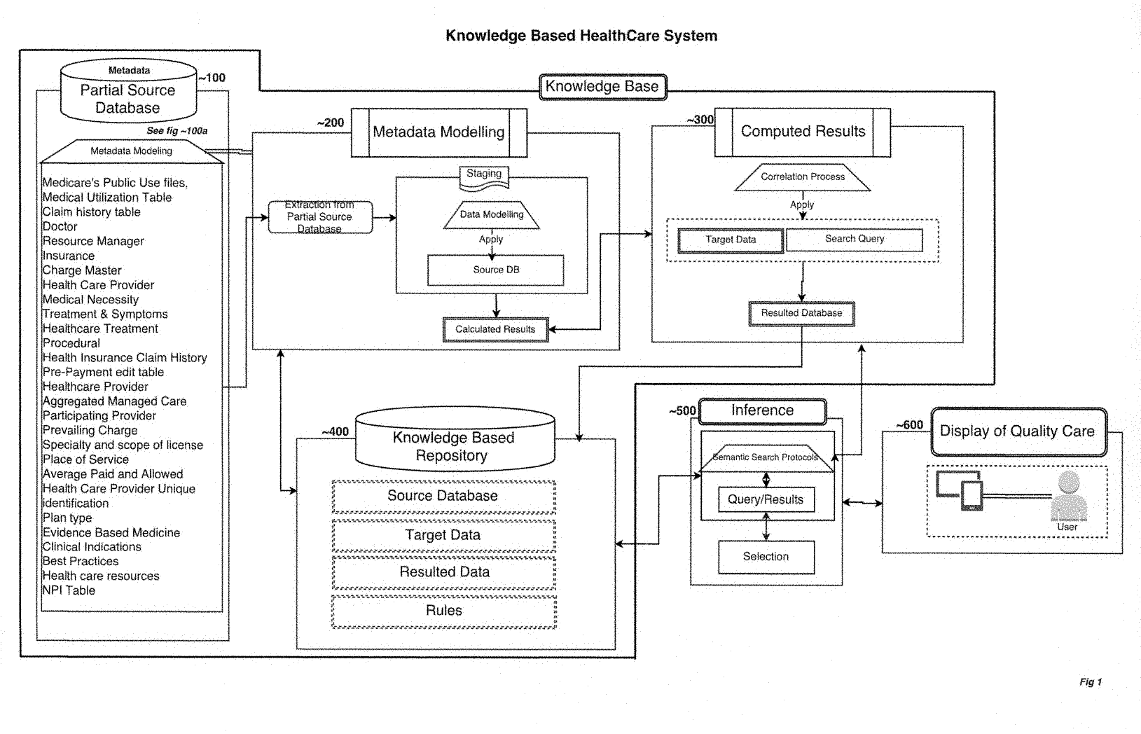

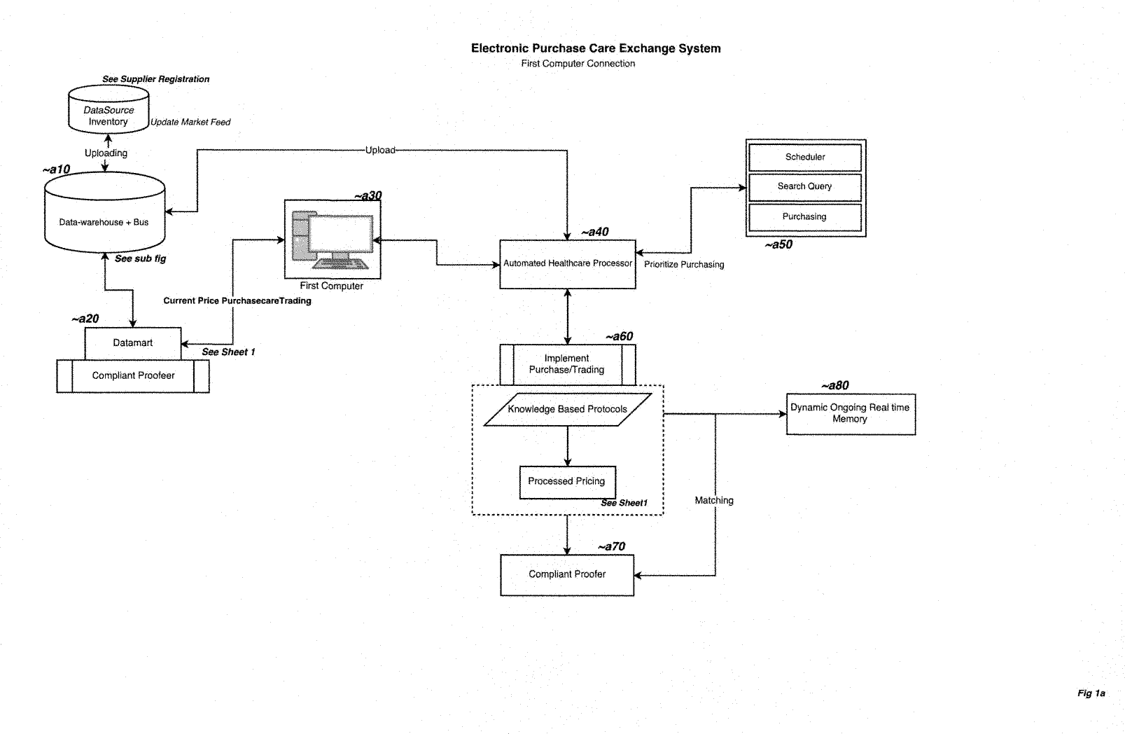

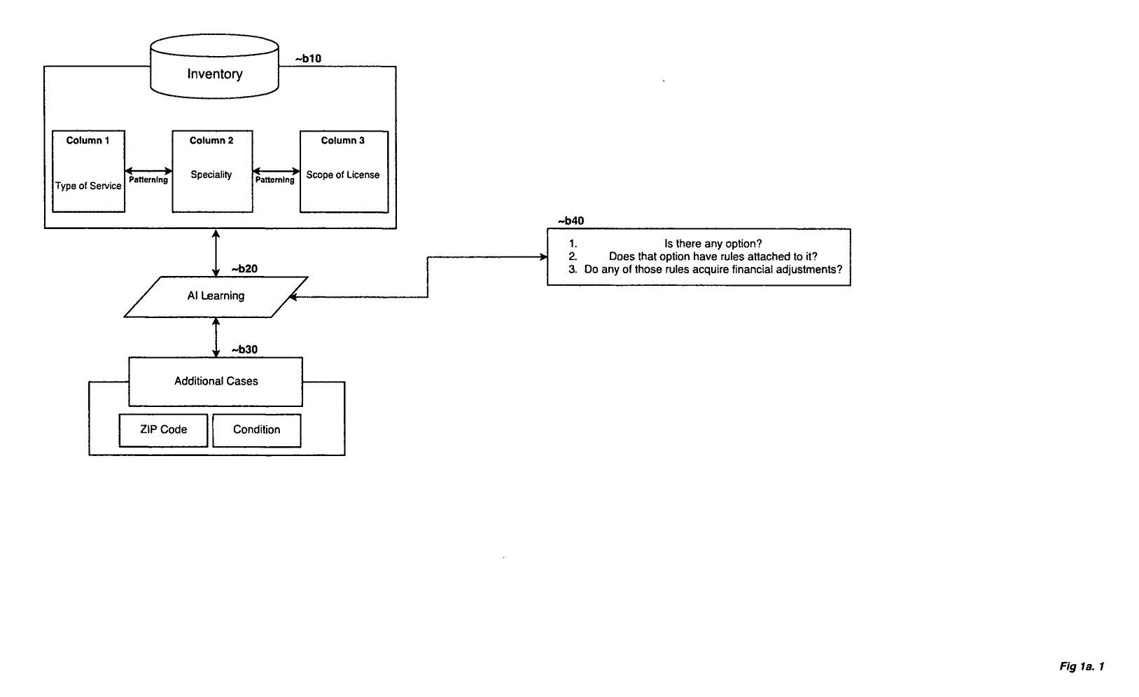

FIG. 1 provides a schematic of an embodiment of a purchase care exchange system network that may be used in connection with the present invention. Other network FIG. 1d arrangements may be used as well. Accordingly, FIG. 1 illustrates a component of an automated purchase care exchange site 100 that incorporates machine learning algorithms and data mining into metadata. Embedded into the metadata 1b are free and ubiquitous healthcare and health insurance industry data that include conditional pricing and other types of protocols, familiar to those in the art; capable of transforming legacy protocols to a plurality of exemplary training examples into training sets to train machine learning algorithms when a healthcare option is payable, non-payable or adjustable Sheet 1.

In one embodiment of the present invention, training set protocols are capable of transforming a bid for a healthcare option to a manufactured compliant proffer. Therewith reducing the risk of overcharges, unnecessary healthcare, fraud and abuse. The knowledge can be stored in a format that's readily usable by a processor 200-400. By example, one class of such data is stored in transaction databases from which all items obtained in a single transaction can be retrieved as a unit. The transaction can then be electronically examined to determine what items typically appear together, e.g., which healthcare items consumers typically buy together in a database of purchase care transactions, such as physical therapy and an office consultation. This in turn gives insight into questions such as how to market these treatments and/or products more effectively, how to group them or product packages on the computer screen Image 1., or which items to offer on a sale Image 1. (promotional screen) to boost the sale of other items. Accordingly, one embodiment can focus on determining which groups of items, called itemset frequently appear together in transactions. From any itemset an association rule may be derived which, given the occurrence of a subset of items in the itemset, predicts the probability of the occurrence of the remaining items. The support of an itemset is the ratio of transactions containing the itemset to the total number of transactions. For example, the association rules can be applied to the data sources for data patterns, and using the criteria support and confidence formulas to identify important relationships. One embodiment of the present invention may use association analysis for discovering interesting relationships hidden in large data sets a10. For example, the following rule can be extracted from the data shown in Sheet 1.

{healthcare option}→{medical condition}

This rule suggests that a strong relationship exists between the purchased healthcare option (sale) of a hemodialysis procedure (CPT code 90935) with a single evaluation by a physician or other qualified healthcare professional and the medical condition Hypertensive heart and chronic kidney disease with heart failure and with stage 5 chronic kidney disease, or end stage renal disease (ICD-10-CM code 113.2) Sheet 2.

Assuming for a moment that the association analysis determined that hemodialysis and the stage 5 chronic kidney disease, the following scenario will itemize a list of healthcare that a buyer wishes to purchase. In this example transaction Ti includes A, C, D (CPT code 90935,90947,83876) Sheet. A review of Sheet 3., illustrates two training sets as defined in Sheet 1; (1) 90935 is incidental to 90947 therefore 90935 is not payable. (2) 83876 is considered not medically necessary for ICD-10 and may not be payable nor reimbursed by insurance. The bid through the electronic purchase care exchange system was $209, however, after applying the training set rules to the itemized list of healthcare options the $209 was repriced to a final compliant proffer of $137.23. A 35% reduction from the $209 bid. Medical billing is eliminated. The confidence level is 2:3, that is 2 out of the 3 ICD-10 diagnosis were the same.

The support of itemset can be derived from the ratio of the count of items purchased together to the total count of healthcare items in all transactions. The coincidence of an itemset is the ratio of the count of items in the itemset to the total of those same items in the database. The dominance of an item in an itemset specifies the extent to which that item dominates the total of all items in the itemset. To illustrate these concepts, an example database is shown in Sheet 2. For the purpose of simplification, in this example, assume that Items A-F represent are unique healthcare options and there are five transactions in the database. The supports of single items are shown in Sheet 4, and the supports of some itemsets in Sheet 4. There are five transactions in the database, so the support is the number of transactions in which the itemsets occur divided by 5. The confidence values of some association rules are shown in Sheet 6. the confidence of 100% for the rule A C means that every transaction which contains A also contains C (see example of itemset concepts & definition of items). Obviously, several algorithms can be proposed for finding generalized itemset from items that are classified by one or more taxonomic hierarchies.

The class of quality of care include multiple variables, such as healthcare option and medical condition, these variables are conceptualized and measured for accuracy, therewith learning algorithms are predicting outcomes, such as quality of care 5a. This training set measures the quality of care Sheet 3. includes examples in table Healthcare Options and Medical Conditions text and Healthcare Options and Medical Conditions with additional columns that include coding conventions.

The core algorithms lie at 200, where machine learning takes place. Good clean data are fed to algorithms 200 and extract knowledge and information. This knowledge can be stored in a format that's readily usable by a machine in the next step. 200 can be used for analysis and construction of models applicable to and useful for predefined classes of problems (see training set).

The data is process 300 to determine any pattern or if there's anything obvious, such as a few data points that are vastly different from the rest of the training set.

Knowledge base repository 400 applies what has been learned in the past to new data. Starting from the analysis of known training datasets (training sets), the learning algorithm produces an inferred function 500 to make predictions about the output values. In one embodiment of the present invention, the system is able to provide targets (classes) for any new input after sufficient training Table 2.

The present invention is capable of comparing within an itemset 5a its output with the correct, intended output and find errors in order to modify the model. The knowledge base may apply reinforcement algorithms 400 that can produce actions such as a compliant proffer, and discover errors or rewards. The machine and software agents automatically determine the ideal behavior within a specific context or instance in order Table 1., to maximize its performance (see compliant proffer). When the EDI 835 data elements are uploaded FIG. 6 (108-114) from which the knowledge base FIG. 1 receives feedback—a reinforcement signal—to learn which action is best. The inference rule engine generates intrinsically new knowledge (500).

The inference and machine learning are combined 500 allowing to re-conceptualize user experiences, depending on the context or instance. By performing various checks of the information used in decision-making and/or information concerning the healthcare purchase order the present invention is capable of validating accurate and compliant purchasing (600).

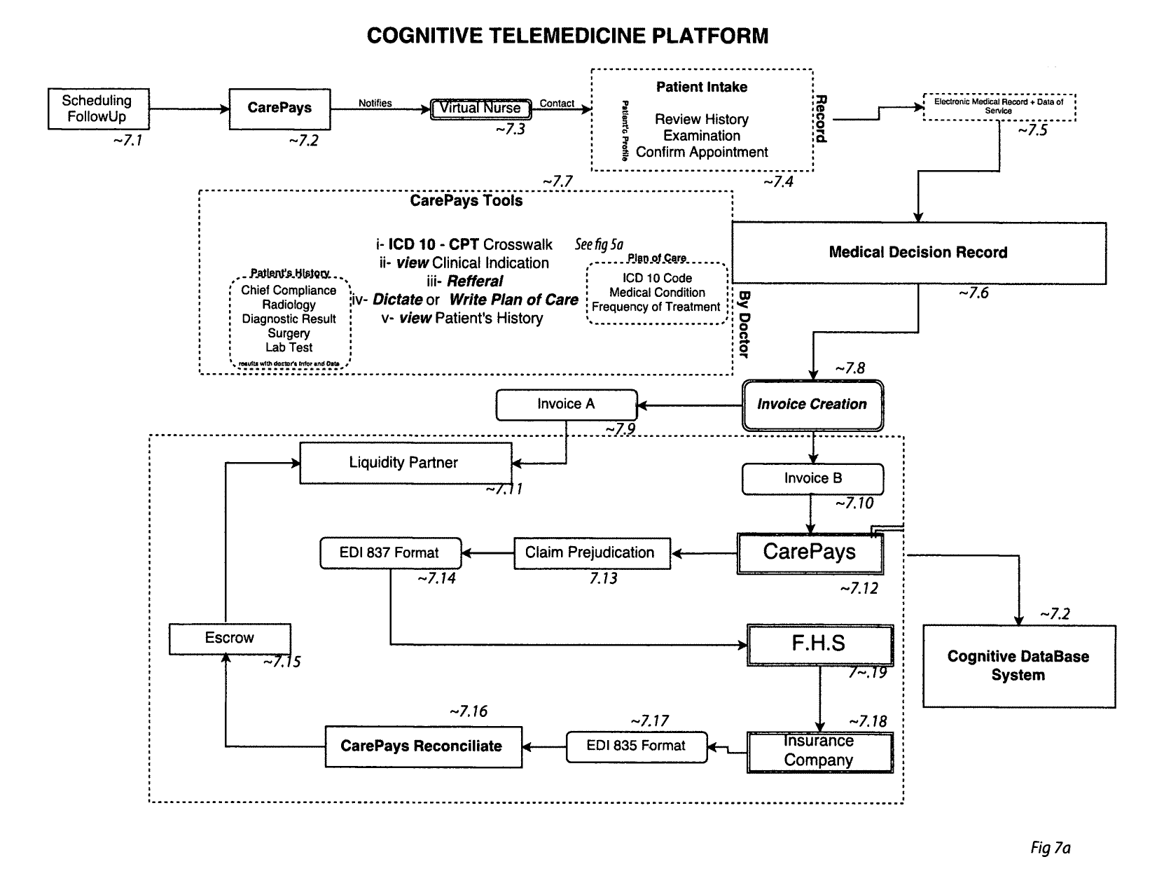

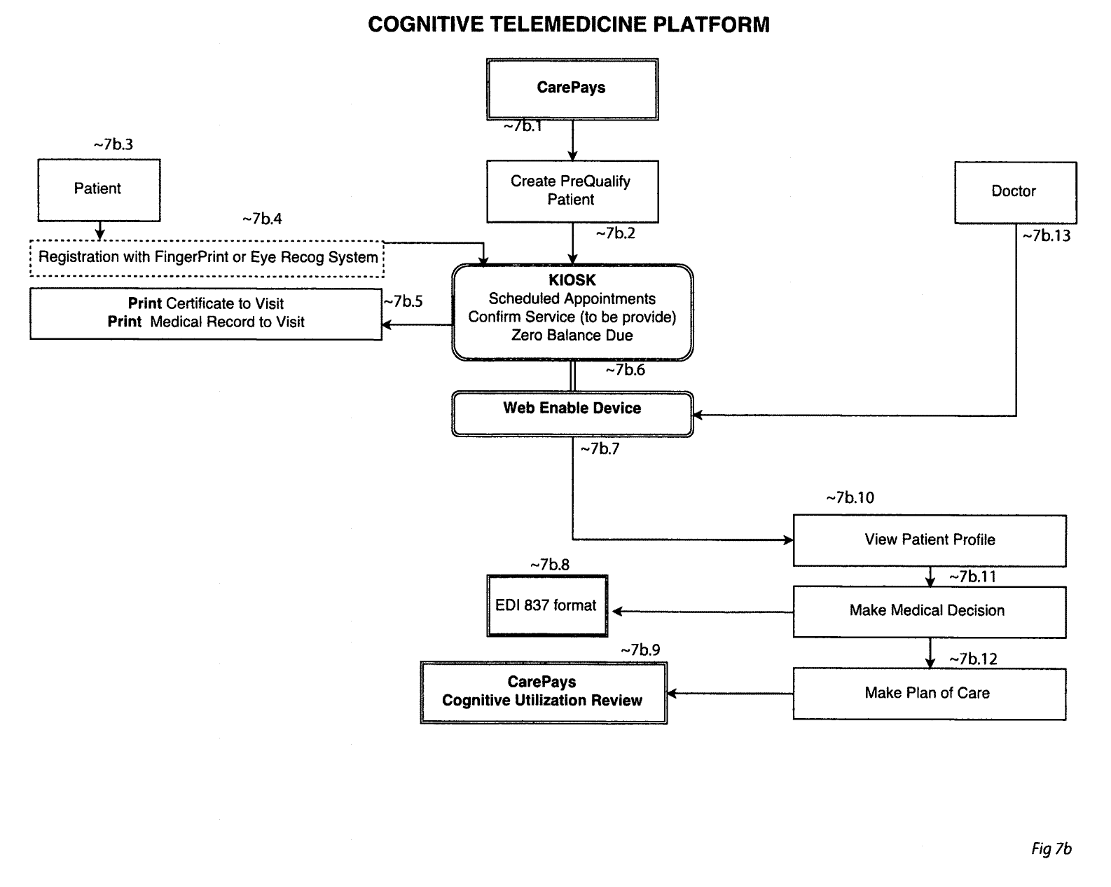

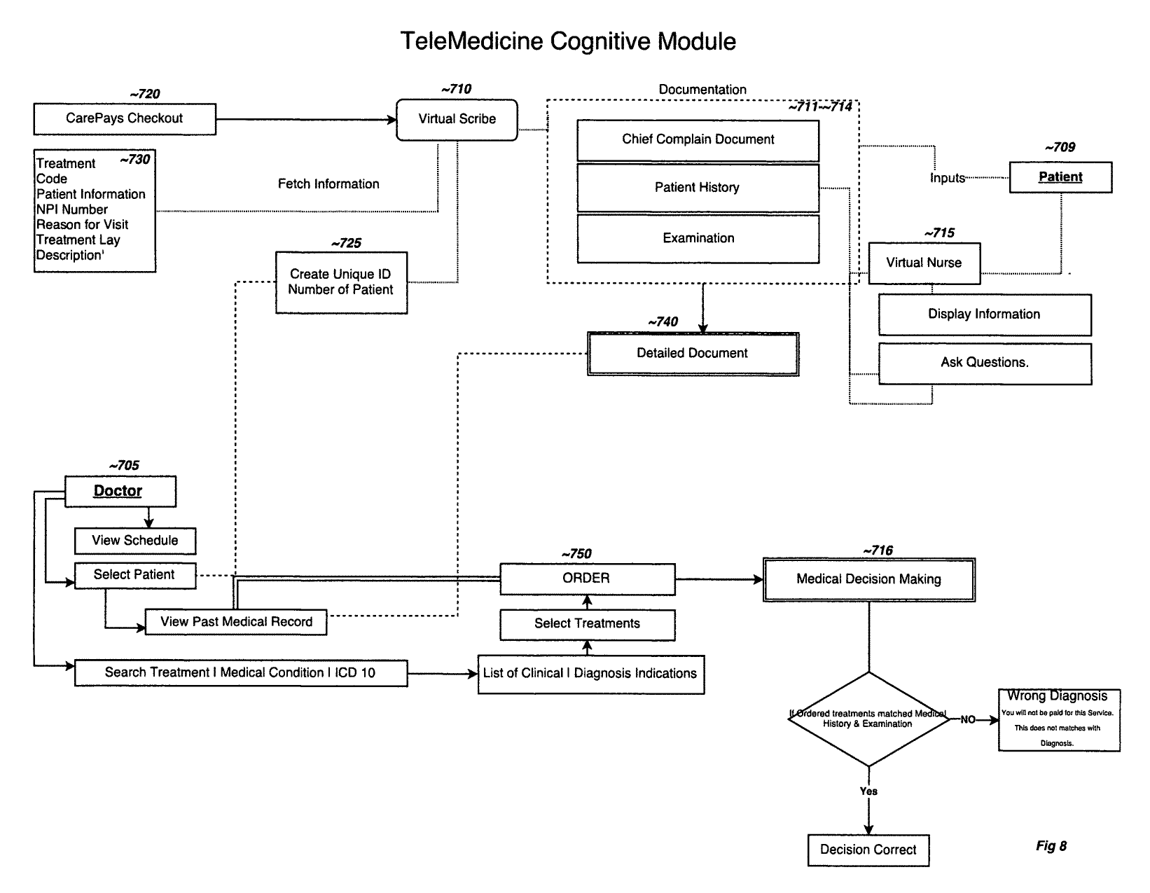

FIG. 1a, a component of the present invention may include an updated inventory market feed. The inventory data source includes one or more supplier agreements and a sale price-range listing of healthcare services and products. Each supplier registrant details FIG. 7.

FIG. 1a further illustrates an automated purchase care exchange site a10 represents a knowledge representation that's acquired from training a machine learning algorithm and data mining. The source data a10 is free and ubiquitous 1b within the healthcare and health insurance industry. Embedded in these sources are plurality of protocols that govern when a healthcare service or product is payable or non-payable or adjustable. Other protocols include clinical indication(s) to establish when healthcare is medically indicated. These clinical and pricing conditions are deemed protocols and are well understood in the art.

The core algorithms lie at a10-a20, where machine learning takes place. Good clean data are fed to algorithms and extract knowledge or information a10. This knowledge can be stored in a format that's readily usable by a machine in the next step. a20 can be used for analysis and construction of models applicable to and useful for predefined classes of problems Table 1.

FIG. 1a, consider the following transaction, a buyer (a30) uses an intelligent device and communication over a network, to connect with an automated purchase care exchange system. The buyer accesses a semantic search engine to select a healthcare service or product. After completing the search, the buyer selects two services, both services are related to kidney dialysis. The buyer wants to purchase a dialysis procedure other than hemodialysis (e.g., peritoneal dialysis, hemofiltration, or other continuous renal replacement therapies), with a single evaluation by a physician or other qualified healthcare professional. The other procedure the buyer wants to purchase is a hemodialysis procedure with a single evaluation by a physician or other qualified healthcare professional.

The present invention is capable of text analysis Table 5., comparing text and numeric values Table 5.1 from previous task Table 1. FIG. 1, the core algorithms lie between a10 and a30, depending on the algorithm. Data is extracted from the data sources and transformed in a usable format. In one embodiment of the automated present invention, good clean data from collected data and prepared input data are extracted knowledge or information.

The present invention is further capable of measuring attributes Sheet 6. that, when combine with other attributes make up a training example. This is usually columns in a training or test set (a10). In one embodiment of the automated present invention, knowledge representation may be in the form of a set of rules Sheet 1.

The present invention is capable of reviewing meta data to see if any patterns or if there's anything obvious. Consider the following scenario, over a period of time the analysis of input data recognizes a pattern between the healthcare service or product and the medical condition or symptoms recorded by the buyer; Probability measurements can be used to develop classification. A class of quality of care can be constructed based on predicting cumulative probabilities Sheet 6.

FIG. 1a Schematic of an embodiment of an electronic purchase care exchange system network that may be used in connection with the present invention. The first computer a30 (intelligent device) is electronically connected to the purchase care exchange system network. Other network arrangements may be used FIG. 1d as well. The electronic purchase care exchange system is an intermediary site and includes an electronic purchase care exchange system and a plurality of actor sites FIG. 1d, for purposes of simplification, FIG 1a illustrates an electronic purchase care exchange system linked to a single site a30. Other actor sites FIG. 1d may be located in a different part of the city, a different country, or different continent as the purchase care exchange system FIG. 1+1a. The purchase care exchange system need not be limited to equipment. The actor sites need not be limited to equipment provided at an actor site, but may include equipment at multiple locations linked by a network, such as a side area network (WAN).

The electronic purchase care exchange system site a10-a80 may be linked to the actor site a30 by one or more communication links FIG. 1d. The communication links FIG. 1d may be part of a wide area network formed by dedicated communication lines, commonly-accessible communication lines, or a combination thereof. For example, dedicated lines may be strung between the electronic purchase care exchange system FIGS. 1 & 1a and one or more of the actor sites FIG. 1d. Alternative, dedicated lines may be leased from telephone, cable, or other communication network operators. For example, the public switched telephone network may embody the commonly-accessible communication lines. Of course, the communication lines FIG. 1d may also include, in whole or in part, wireless communications, such as microwave or satellite links.

In one embodiment of the present invention, the purchase care exchange site may be linked to a purchasing site or buyer by one or more communication links FIG. 1d. The communication links may be part of a wide area network formed by dedicated lines, commonly-accessible communication lines, or a combination thereof. For example, the dedicated lines may be strung between the purchase care exchange site and one or more of the member sites FIG. 1d.

The present invention is capable of reducing the time it takes for the buyer to submit a buyer order, indicating types, quantities, and bid prices for healthcare services and products or quote in response to incoming purchasing information from the electronic purchase care exchange system. In accordance with one aspect of the present invention, the electronic purchase care exchange system automatically decides whether or not to process a buyer order line item or make a compliant proffer Sheet 3 (quote) based on protocols and decision logic, and purchasing information received from the electronic purchase care exchange system computers. To decrease the response time, the automated electronic purchase care exchange system may be dedicated or substantially dedicated to performing automated purchasing operations, with limited or minimized overhead permitted for other tasks unrelated to purchasing. The present invention is further capable of reducing the time delay due to network lags arising from the transfer of purchasing information from the electronic purchase care exchange system computers to the intelligent device, and vice versa FIG. 1.

In one embodiment, the electronic purchase care exchange system FIGS. 1 & 1a may be designed as a local area network (LAN) and include, for example, or ore more security routers and one or more back office computers, among other equipment. For purposes of illustration only FIG. 1d, two security routers (6.14 & 6.13) and three back office computers (6.12) (referred to collectively as back office computer 6.1 are shown in (FIG. 1d). The security routers), (6.13 & 6.14) and communication links (6.11). each security router (6.13 & 6.14) transmits and receives commutations over the communications links (6.11), as well as restricts communications from unauthorized sources. More particularly, the security router (6.2), (6.3) may be used to isolate the equipment at the electronic purchase care exchange system site FIG. 1 from intrusion and facilitate communication with the back-office computers (6.1).

The actor sites may include a LAN architecture having one or more security routers, one or more backend computers, one or more actor stations, one or more hubs, among other equipment. For purposes of illustration only, FIG. 1d shows two security routers (6.13 & 6.14), two backend computers (6.4 & 6.12) three actor sites (6.6), (6.7), (6.10) (collectively referred to as the actor sites) and two hubs (6.5) (6.9). The security routers (6.2), (6.3) transfer transactional information between the actor site (6.4) and the electronic purchase care exchange site FIG. 1. and screen communications from unauthorized sources. The hubs (6.9) distribute data between the backend computers (6.4). distribute data between the backend computers (6.4), (6.12) and the actor sites (6.6).

Backend computer (6.4) may be configured as a communication server for the actor sites (6.10, 6.7 & 6.6). The electronic purchase care exchange system may supply software and/or hardware for the backend computer (6.12) to facilitate communications with the electronic purchase care exchange system FIG. 1. Backend computer (6.12) may also be equipped with software and/or hardware that facilitates communications with the electronic purchase care exchange system site.

One embodiment of the present invention, a healthcare option Table 1a. stores information concerning healthcare options that may be automatically purchased. For simplicity, a two-dimension table having rows and columns will be described. However, it should be understood that higher dimension arrays or tables may be used in connection with the present invention. Each row of the healthcare option table (need #) stores information relevant to a particular option include for example, healthcare care option, a supplier's average charge for a healthcare option, the average bid amount, a supplier's current reserve price, a current spread, total compliant proffer, current target price including place of service inputs that may be needed to calculate healthcare option transaction prices. As discussed below, this information may be used as a check against erroneous operation. Alternatively, the healthcare option data table (Example 3) may store information in connection with items that are actually being automatically purchases at a given time. As a further alternative, healthcare option data table Sheet 5 may store information in connection with all of the healthcare options that may be subject to automated purchasing and include indices that link only the healthcare option items currently enabled for automated purchasing. Accordingly, any search of the healthcare option data table can skip those entries for which automated purchasing is not enabled. In such a case, an additional option data table may be maintained for the full set of items for which automated purchasing may be performed Image 4. This is useful in increasing the speed at which a disabled healthcare option can be enabled. Accuracy checks may use both the additional healthcare option data table and the healthcare option data table Sheet 5. Communications between the automated purchase care exchange system and the actor sites 1a are conducted through an actor site interface a10, a30. For example, an actor site may update information contained in the healthcare option data table via an actor site interface a30. In this way, the healthcare option data table Sheet 5 may be updated to enable (disable) automated purchasing for a particular healthcare option.

The healthcare option data table Sheet 5 may be organized in several different ways. For example Sheet 5, the average bid amount and average compliant proffer for a particular healthcare option may be stored in different rows of the healthcare option data table Sheet 5. alternatively, the average bid amount and the compliant proffer prices may be stored in the same row of the option data table sheet 5, but in different columns, or as different cells in a price dimension, for example. Also, the healthcare option data table Sheet 5 may be segmented, for example, so that all bid prices are grouped together, and all compliant proffers are grouped together Sheet 7. Different classes of options (i.e., healthcare options with different underlying securities) may be indexed in a single data table sheet 7 or in multiple look-up table sheet 7.

In addition to the current market information concerning a healthcare option purchase, the automated purchase care exchange system may receive and decode current market information concerning the security (or securities) underlying the healthcare option Sheet 5. For example, an electronic purchase care exchange system that sells the underlying security typically would maintain an inventory a10 & sheet 5. The automated electronic purchase care exchange system network determines whether a buyer order or quote should be submitted based on, for example, the current market price of a healthcare option and the buy and sell prices sheet 5. The predetermined market prices Sheet 5 (see healthcare option column) of the underlying security are derived from the inventory feed sheet 5. The compliant proffer price is derived from inventory, among other things 1b, the current market price of the security underlying the healthcare option. The buy and sell prices are calculated when underlying factors, such as protocols, that contribute to the prices are satisfied. Computational times of the prices may be reduced by using pre-calculated sheet 3 values and/or using interpolation and extrapolation. Other techniques may be used in addition or in alternative to speed automatic decision making. In addition, a system of checks may be conducted to ensure accurate and compliant purchasing.

The price logic is tasked with generating the prices based on input information. The price logic may be implemented in hardware or a combination of hardware and software. For example, price logic may be implemented by the central processor and memory a10, and possible other equipment useful in perforating fast mathematical calculation in a general purpose computer. Alternatively, the price logic may be implemented in a separate processor in communication with the processor of a general purpose computer or an array of processors FIG. 1. Of course, theoretical price logic may be embodied by other devices capable of generating prices Sheet 3 as described above.

The price logic generates prices in accordance with price adjustment protocols sheet 3 (see instance column) and in accordance with mathematical models. The combination of conditional pricing and other protocols are factors that influence the mathematical models that produce a theoretical value for a healthcare option value input variables that may change over time. Healthcare option pricing input variables considered in these models may include (1) the current market price of the underlying security (e.g., the price of the healthcare option from which the healthcare option is derived), (2) price adjustment, (3) payable healthcare options, (4) non-payable healthcare options, and (5) protocols.

The current market price of the underlying security of the healthcare option may be defined in several different ways. at any given time during the purchasing, the underlying security will usually have (sheet 5): (1) a bid prices and quantities; (2) compliant proffer and quantities; (3) a last paid price and volume at which the underlying security was purchased (last paid to supplier type); (4) zip code of supplier; (5) supplier type; sheet 7; (6) an average of the current highest bid and the lowest compliant proffer (best bid, best compliant proffer) sheet 5; (7) an average price of a certain depth Sheet 5 among other values, and (8) spreads may be used to calculate healthcare option transaction prices such as a compliant proffer. Obviously, there are many more definitions of underlying price that can be created, for example, using permutations of the seven definitions provided above. In summary, value calculations used for purchasing may use any of several definitions of underlying price (Sheet 1).

In another embodiment of the present invention illustrated in sheet 5 the paid and inventory sale price table (need #) stores healthcare option transaction prices, for which purchasing was performed. Of course, the price table sheet 5 may be formed as part of the same table as the healthcare option data table. (example 3) ART NOTE: EXAMPLE 3 SHOWS A COMBINATION OF PRICE TABLE AND HEALTHCARE OPTION.

Similar to the healthcare option data table sheet 2, the price data table may be organized in several ways. For example, all healthcare option paid prices for a given set of healthcare option pricing input variables (1)-(8) may be provided in a single column of price data table, with a separate price data table provided for compliant proffer (example 3). Alternatively, the price data table sheet 2, may index both a buy price (paid) and a compliant proffer price. The price data table (example 3) may be segmented or multi-dimensional. Moreover, the price data table may be combined with, for a portion of, or be linked to healthcare option table sheet 2. The values coupled with protocols in price data table may be compared with market healthcare options prices sheet 5 and may trigger automated purchasing buy and sell decisions between the buyer and the electronic purchase care exchange system. While the price data table (example 3) may take many forms, a table like the healthcare option data table (UR) with each row representing a single healthcare option will be described for purposes of simplicity. As noted above, it should be understood that data structures other than tables may be used in connection with the present invention.

In addition, the healthcare option data table sheet 2 and the price data table sheet 5 can be structured consistent with the particular search protocol used by the table update protocol sheet 1 so that certain healthcare options or other items are located by the updated protocol before other options or items. For example sheet 5, if table update protocol implements a linear search, the contents of healthcare option table sheet 5 for options at the top will be updated before options at the bottom even with multiple rows need updating. If the change in content affects the healthcare option transaction price stored in the price data table sheet 7,8 this implies healthcare options near the top could be recalculated and compared against market healthcare options prices sheet 5 before healthcare options at the bottom of price data table sheet 7,8 Accordingly, the electronic purchase care exchange computer may structure the healthcare option data table sheet 7,8 and/or the price data table sheet 7,8 so that healthcare options that have shown in the past, or are likely to show in the future, the most promising profits will be located first. The particular order of the healthcare options in the data tables sheet 7,8 and sheet 7,8 may depend on the purchasing volume in a healthcare option, for example Healthcare options with relatively high purchasing volume over recent purchasing days or the current purchasing day may be given a higher priority rating in healthcare option table sheet 7,8 and/or price data table sheet 7,8.

Accordingly, structuring the healthcare option data table sheet 7,8 and price data table sheet 7,8 as described increases the opportunity for the buyer to participate in the most lucrative transaction when there when there are restrictions on the number of concurrent purchased orders placed. In addition, or in the alternative, the price logic sheet 1 may calculate the prices for healthcare options likely to yield the highest profits. The calculated prices may be supplied to the decision logic (knowledge base) either before the price logic calculates the price of another healthcare option or concurrently therewith.

In accordance with the embodiment shown in FIG. 1, the automated purchase care exchange system may respond to changes in market conditions by changing any of the healthcare option pricing input variables (1)(8), changing sell spreads, or changing any other variables that might affect healthcare option transaction prices. These changes would update the appropriate values for each healthcare option of the healthcare option data table sheet 7,8 and then, since the changes affect healthcare option transaction prices, would trigger a recalculation of the compliant proffer in the price data table sheet 5. Of course, updates to healthcare option data table may originate directly from the automated purchase care exchange. For instance, healthcare option pricing input variable (1), the price of the underlying security, used in healthcare option pricing models (see training sets) may receive dynamically from the present invention. In this case, healthcare option data table sheet 7,8 and subsequently, price data table sheet 7,8 would be updated automatically in real time with no intervention during the purchasing from the automated purchase care exchange intervention.

The time needed to calculate these values may depend upon the healthcare options specifications, the particular protocol, and mathematical model used to calculate the value, the use of pre-calculation short cuts, and the level of desired precision of the calculated value. Of course, various mathematical models may be used. Obviously, hardware is also an import consideration, as faster and more efficient computers tend to reduce calculation times. The healthcare option specifications and each of the price data models are known in the art and will not be described here.

In one other embodiment of the present invention price assurance referring to image 6, decision logic (knowledge base is a knowledge representation) compares the compliant proffer calculated and stored in the price data table to market price for the healthcare option and, based on the comparison, determines whether the healthcare option should be purchased from the inventory market feed or sold to the buyer, or no action should be taken. For example, in an embodiment in which the price data table sheet 5 stores the paid prices and compliant proffer prices for a particular healthcare option, decisions may be triggered when: (1) a healthcare option price in data table sheet 7,8 changes, but the market bid or compliant proffer price of the healthcare option remain the same, (2) the market bid or compliant proffer price of the option changes, but the prices in a data table sheet 7,8 remains the same, (3) automated purchasing is enabled for a particular healthcare option, and (4)

Consider example sheet 5 in which a price of a particular healthcare option stored in price data table sheet 7,8 changes and the bid and compliant proffer price of an option remain static. As noted previously, the price data table sheet 7,8 may be updated when one or more of the values that affect the buy and compliant proffer prices changes such as, but not limited to, the buy and compliant proffer spreads and/or healthcare option pricing input variables (1)-(8). For example, healthcare option input variables (1)-(8) discuss previously could change, perhaps due to a change in the present invention's assessment of market conditions. These changes may occur when buyer enters new information through an intelligent device a30 or when new information becomes available through another source (example 3. Art Note: New information can include NCCI edits plus medical necessity criteria). A change in one or more of the healthcare option pricing input variables (1)-(8) triggers a re-computation of (probably) all values in the price data table (example 3). As previously noted, re-calculation may involve re-calculating the price data in its entirety or using pre-calculated values, extrapolation, and/or interpolation. For purposes of discussion below, assume healthcare option pricing input variable (1), the price of the underlying security, changes and thereby changes a bid or compliant proffer price of a particular healthcare option in the price data table (example 3). Decision logic (knowledge base) a60-a80 will compare the current market charge price of the healthcare option to the new compliant proffer price obtained from the price data table (example 3). In this case, the decision logic a60-a80 performs all comparisons affected by the change in the underlying price. Accordingly, the decision logic (a50-a80) makes comparisons with market bid or compliant proffer prices corresponding to new inventory and paid/buy prices.

Consider example (2) in which the market bid price for a particular healthcare option changes and the prevailing prices in the data table of the healthcare option remain constant. The decision logic a60-a80 will compare the new market bid price to the corresponding paid price that exists at that time from the price data table sheet 5. Accordingly, a change in market bid price of a particular healthcare option may trigger a comparison of market bid price to theoretical compliant proffer price. Based on the comparison, for example, if the market bid price is greater than or equal to the compliant proffer price, the automated purchase care exchange system may prepare a response (such as an order or quote) for the particular healthcare option.

Consider example (3) when automated buying for a particular healthcare option is changed from disabled to an enabled state. This can occur when a healthcare technology was determined to be experimental or investigational, non-payable. Using data mining techniques, a relationship was established between a healthcare option and an input variable. Over time, the values of this particular healthcare option was changing from zero dollars to dollars being paid. The correlates indicated that the market was willing to purchase this care. The source of the payment data came from uploaded EDI 837 FIG. 6 or explanation of benefits (the insured market) and bid data stored in the price data table stored in memory. Over time, the mathematics indicated a significantly higher probability of payment from a variety of source data. Therewith, present invention's learning led to a behavioral changed from indicating that the non-payable healthcare option should be considered for purchase (i.e., payable). The instances that resulted in the behavioral changes including the price data table changes were stored in knowledge base and metadata memory sheet 1.

In addition to enabled and disabled states (example above), a third, “swarming up” or “test” state may be provided for a healthcare option in the automated purchase care exchange system. In this third state, the electronic purchase care exchange system may perform all steps except actually placing an order. This allows the present invention to monitor the operation of the automated purchase care exchange system without actually submitting orders, thereby reducing the risk of enabling healthcare options for automatic purchasing using price data which are not market realistic.

Consider example sheet 1, a conditional price check condition relating to price adjustments increased via a command from an intelligent device. In connection with the conditional pricing, the entire transaction may be held in a “pause” state if it had made more than a predetermined number (e.g., 3) automated bid attempts within a predetermine period of time (e.g., 60 seconds). If this conditional price checker is disabled or relaxed, for example, by increasing the predetermined number of attempts (e.g., from 3 to 5) the conditional pricing check may no longer be in violation. As a result, the entire automated purchase care exchange system may transition from the “paused” state to the enabled state. If a particular healthcare option has been enabled for automated purchasing, the decision logic (knowledge base) will then compare the market bid price to the compliant proffer price in table.

Of course, the automated purchase care exchange system may be designed to automatically switch from the “enabled” to “pause” state if conditions are deemed too risky to run an automated purchase. For example, the automated purchase care exchange system may change from an “enabled” state to a “pause” state when it senses, or receives a message, that (1) communication in any of the communication links is not working properly FIG. 1d, (2) the difference between compliant proffer and bid price of the underlying security is greater than some predetermined value, (3) the rate of change of the price of the underlying security is greater than some predetermine value, (4) and/or (5) release of know news events is pending. Assume the system automatically went into the “pause” state due to one of the conditions above. The automated purchase care exchange system can be design to either automatically go into the “warm-up” state when the triggering condition has passed, or require manual intervention to move from the “pause” to the “warm-up” stage.

Decision logic a60 determines that a bid price order should be submitted if the market bid is greater than or equal to the price target. Even if decision logic a60 determines that a buyer order should be submitted, the conditional price checker logic a60 may be used to prevent a purchase care order from being submitted or paused. The conditional price checker logic, for example, can generate warning alert notifications when: a bid may have additional charges associated with it, for instance, the use of a co-surgeon during a scheduled surgery, or when a secondary bid is payable or non-payable, or when a medical facility charge may be added a surgery bid, or when a medical service may not be considered medically necessary, or when a plan of care is inconsistent with healthcare industry standards, or if insurance is involved, a notification alert indicating to the buyer that their insurance may not reimburse the healthcare option, all of these protocols and those listed on the training list are familiar to those in the art, and/or issuing a notification alert before the scheduled date of service, to a remote intelligent device that informs the purchaser before the scheduled service date that a lower price is available. Also, the automated purchase care exchange system may be paused or stopped if the number of attempted orders during a transaction event exceeds a predetermined amount in a predetermined period of time. The constraints may be stored in the healthcare option data table (use example 3 and supper coder) or elsewhere and may be varied for individual healthcare options. Other constraints may involve generating warnings and or/preventing orders, for example, when: (1) the bid price is less than the price target, (2) the bid price exceeds the price target, sheet the price target is greater than the compliant proffer price, (4) the price of the underlying security moves outside the price range stored at inventory for a particular healthcare option, (5) the compliant proffer prices is less the compliant proffer price plus spread.

If conditional pricing is passed, order logic 500 (inference) creates a response and submits the response to the electronical purchase care exchange system FIG. 1 site via an output interface. The intelligent device may be notified through an electronic purchase care exchange system interface whether or the conditional pricing checks are passed. The intelligent output interface a60 may pass the order to the electronic purchase care exchange system interface software for ultimate transmission to the electronic purchase care exchange system FIG. 1. The receiver interface and the output interface may be formed by common equipment and/or data ports.

The healthcare option data table sheet 7,8 and the price data table can be checked periodically to ensure the accuracy of its content. Mathematical formulas, such as correlates can be used to measure relationships and accuracy of the content. Additionally, using machine learning and data mining may be performed every say, 15 seconds to discover and/or develop training sets and populate new learning to knowledge base and metadata. of course, other or additional techniques for testing the accuracy of tables sheet 7 and sheet 8 may be implement. Moreover, such an accuracy check may be omitted if one is sufficiently confident in the reliability of the software, hardware and communication networks.

Knowledge of how the search protocol locates data within the healthcare option data table sheet 7, and the price data table may be used to structure these tables to ensure that selected healthcare options will be located particularly quickly. The selected healthcare options may be, for example, frequently purchased healthcare options and/or healthcare options whose price target will become attractive with a small change in the underlying security price. For example, the search protocol may conduct searches by starting at the first row of the data table and then stepping through each successive row until a particular row is identified. In this case, the tables (example 3) and (example 3) may be structured so that a select healthcare options is placed in the first row. Consequently, the search protocol will locate the select healthcare option first. Statistics may be maintained, for example, at the electronic purchase care exchange system FIG. 1 and used to restructure the tables sheet 5 and sheet 7,8 as purchasing conditions change. Further, when the market price sheet 5 of the underlying security changes, the price logic may reprice the new price target in the same predetermined order as the search protocol, with newly repriced price target acted upon by the decision logic (knowledge base) 400 either before or during repricing of the next target price. In this way, the automated purchase care exchange system reprices theoretical value and makes transaction decisions first for healthcare options believed to be most likes to generate profitable transactions, whether the decision logic 400 is triggered by a change in market price (UR table) of the healthcare option, by a change in price target value, or otherwise. ART Note: Column 1 healthcare service & column 2 medical condition show how when combining an with NCCI table, based on the statistics the table was restructured

The embodiment described in connection with sheet 5 compares the current market price of a healthcare option to a bid price and a sale price from the inventory feed from a price target table (example 3) to rationalize a compliant proffer price decision. However, other transaction values may be compared consistent with the present invention to generate payable and non-payable decisions. For example, the healthcare option value (prevailing charge) may be subtracted from the market bid price and compared to the compliant proffer price (buy) spread selected by the present invention to generate a purchase opportunity (buy healthcare option at compliant proffer). Alternatively, implied instances may be repriced for market healthcare option bid prices using, say mathematical models or statistics including inputs similar to those used for repricing healthcare option prices. The repricing implies instances or conditions that may then be compared to the present invention values to make compliant proffer decisions. Of course, other values may also be indexed and used for comparison to generate purchasing opportunities via a generated compliant proffer a unique decision sheet 3 consistent with the present invention.

FIG. 6 illustrates an embodiment of the purchase screen Image 4 displayed on an intelligent device 600 in connection with purchasing healthcare options on a particular healthcare security or commodity. The purchasing screen 600 may provide a graphic user interface to enable the electronic purchase care exchange system to set parameters associated with automated purchasing. Purchasing screen (shopping window) is organized as a array of comparative shopping windows Image 4. The shopping window displays represents suppliers within a radius, all of whom are qualified and credentialed to render the healthcare required, all of whom are registered as suppliers, each supplier's predefined market price for a healthcare option, the display further includes expected reimbursements from benefit plan (if insured), an automated purchase can be made by purchase now that includes a 10% savings from current market charges for the healthcare option. Or, there is an option to negotiate with the automated purchase care exchange system “name your own price” from the window display. If the bid is below the current market value for a particular healthcare option (See Image 3) an alert notification is issued on the screen with the choice of resubmitting an updated negotiated directly to an electronic purchase care exchange system. There is a sorting feature that allows for the sorting of price or distance etc. The purchasing screen is further organized to enable scheduling of the healthcare option. In one embodiment of the present invention a risk analysis is made based on the credit worthiness and benefit coverage.

The purchasing screen may be scrolled up or down to view additional information, if any exist. FIG 1a Data Source Inventory, b10 are comprised of supplier registrant agreements with a sale price range for healthcare services and/or products coupled to the agreement. After the supplier registration process FIG. 7 has been completed and agreement and purchase order is accepted by the supplier (seller) the agreement plus details to the purchase order are uploaded to inventory “data source inventory”. The present invention every 15 seconds re-evaluates the purchasing activities at 1b. Other strategic procurement activities include analysis, source selection, cost modeling, negotiations, and initiatives that build sourcing value.

Sheet 5 further illustrates an embodiment market driven data. The rows (need #) of the array represent different healthcare options available in the market for the particular security or commodity. The window display of sheet 7 of the array provided information concerning the healthcare options. More particularly, the display windows provide information on healthcare options, price, purchase now, or negotiate price Image 2,3.

Changes made to assumed instances on automated purchase care exchange system FIG. 1 may update healthcare option data table and price data table, and consequently, trigger a repricing of price data table. Values that may also be adjustable in groups, for example a bundle of healthcare services and/or products related to a diagnosis or symptom or a treatment plan or plan of care over a period of time. Another example, the automated purchase care exchange system FIG. 1a may receive an updated inventory market feed providing price information concerning the underlying security of a particular healthcare option. The price information may be used to update or refresh the purchasing screen 600. This may include displayed windows (need #), and values for a given underlying price.

FIG. 6 provides an exemplary progression of steps from transmission of the current market information from the automated purchase care exchange system to receipt of purchasing confirmation by the present invention FIG. 6a. The progression of steps illustrated assumes that the electronic purchase care exchange system required interface software runs FIG. 1 on computers.

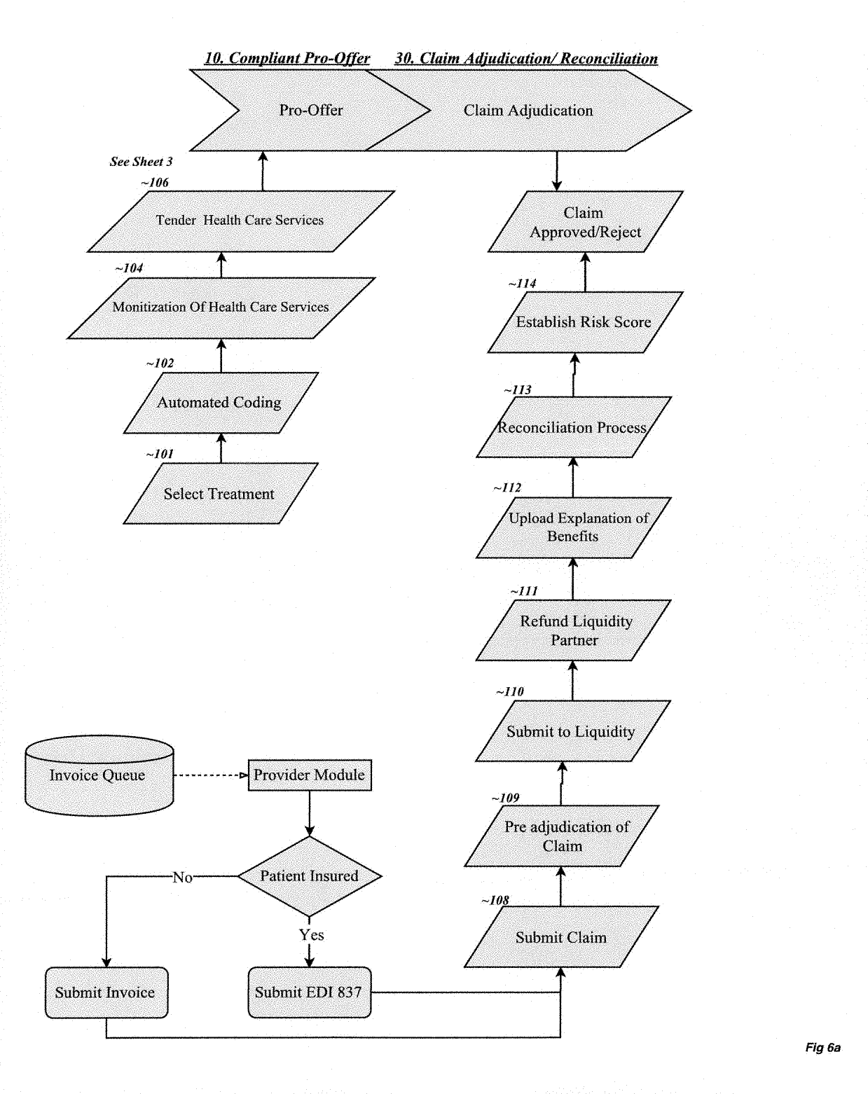

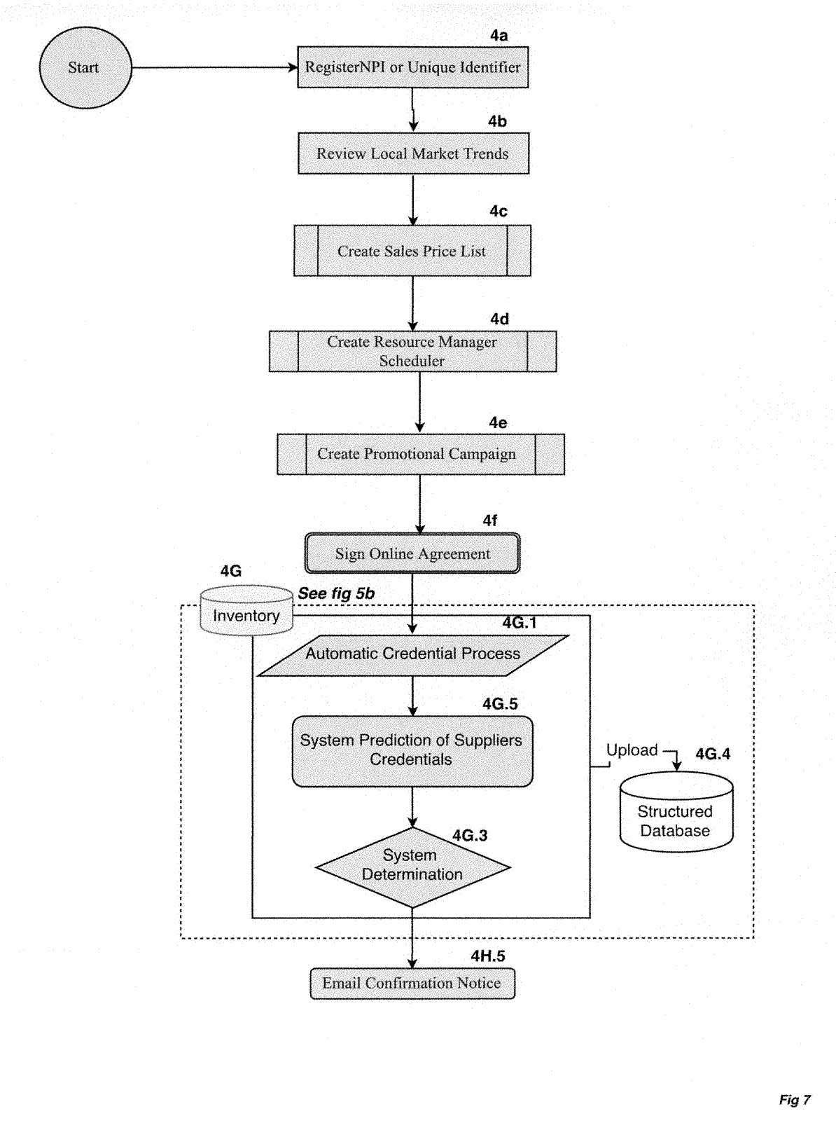

FIG. 6 provides an exemplary progress of steps from transmission of current market information from the automated purchase care exchange system FIG. 1, to receipt of purchase information from the present invention. FIGS. 5 & 6a, a semantic search engine 101 can be used to search healthcare by diagnosis or symptoms or healthcare description, or procedural and diagnostic codes, or by medical specialty, place of service, coupled to zip code image 7; 102-103 may include a unique intelligent healthcare option sheet, said sheet changes based on a particular healthcare option or diagnosis and/or symptom, which may include additional specific healthcare services and/or products that are associated with a healthcare option. For example, if a surgery is being purchased, based on machine learning and data mining, the present invention is capable of identifying additional cost associated with the particular healthcare selection. In this example, the surgery cost may not include the cost of an anesthesiologist and/or facility fee.

Once the healthcare option(s) have been selected 104 comparative shopping window displays are viewable from the intelligent device 600. The shopping window displays items that are uniquely linked to a particular healthcare option. For example, a row of detail from the market data may accessible; qualified supplier data table can be accessed to display which qualified supplier, within a particular radius, renders the care required and what is the healthcare cost (market price) for each supplier identified from a row of data; the market data is dynamic and updated every 15 seconds. For example, the database structure of the qualified supplier data table may be expanded to include columns in a database, such as a row of information that may include supplier type, address, zip code, credential, gender, what type of place of service the particular healthcare option was rendered, the charge for the healthcare option, the average charge, the average bid, the compliant proffer, the average compliant proffer, the current target price, the average target price, the frequency of a particular healthcare option rendered by a specific supplier; if insurance is involved the amount allowed by benefit plan, the average amount allowed, the paid amount for the particular healthcare option, the average amount allowed by plan type benefit plan; the out of pocket cost for a particular healthcare option, the average out-of-pocket cost for a particular healthcare option, type of conditional price rules that apply to a particular healthcare option, the average paid after price adjustments for a particular healthcare option, the diagnosis and/or symptoms coupled to the healthcare option, the place of service of the particular healthcare option, referring suppliers' linked to particular healthcare option. Obviously, additional training sets can be added to a row of data relative the particular healthcare option. In one embodiment of the present invention an index can be used to significantly speed up data retrieval operations.

From the shopping window 104, a bid is submitted to the electronic purchase care exchange system, the next step is a system of rules (protocols) that audit each transaction for compliance, the application of the protocols transforms a bid to a compliant proffer 10., 600. The transaction is populated a10 with knowledge representation in a60-a70.

The compliant proffer 105-106 and a list of payables are viewable from the cart (show picture). From the cart, if insurance benefits are involved, current coverage including financial obligations with expected insurance reimbursements details are optional. If the verification of health benefits selected, current expected reimbursements and current out of pocket costs are applied to the payable details from the cart. An updated summary of the transaction is generated with the updated details. A more accurate analysis is given based on the current status of healthcare benefits.

FIG. 5 illustrates a scheduler 14-20, one embodiment of the present invention coordinates appointment scheduler between the present invention and the supplier. A reminder notice is electronically sent, before the schedule date of service is sent to a remote intelligent device. If a lower price for a healthcare option is available before the scheduled appointment date, an alert notice is generated by the intelligent device informing the user that a lower price is available before the scheduled appointment date. The intelligent device enables the user to view the details from the intelligent device.

FIG. 6 connects funding sources 3a to purchase the care. The present invention is capable of conceptualizing if health care benefits are involved with the purchase. Based on the class of insureds and metadata one embodiment can process eligibility, authorizations, coverage details, and other related information linked to the healthcare option using EDI transaction sets 108-111 between EDI partners and with an automated purchase care exchange system and scrap the data elements then populate the details to reconcile transactions, to update the healthcare option data table and price data table, including payable and non-payable healthcare based on coverage constraints or compliance issues FIG. 6a.

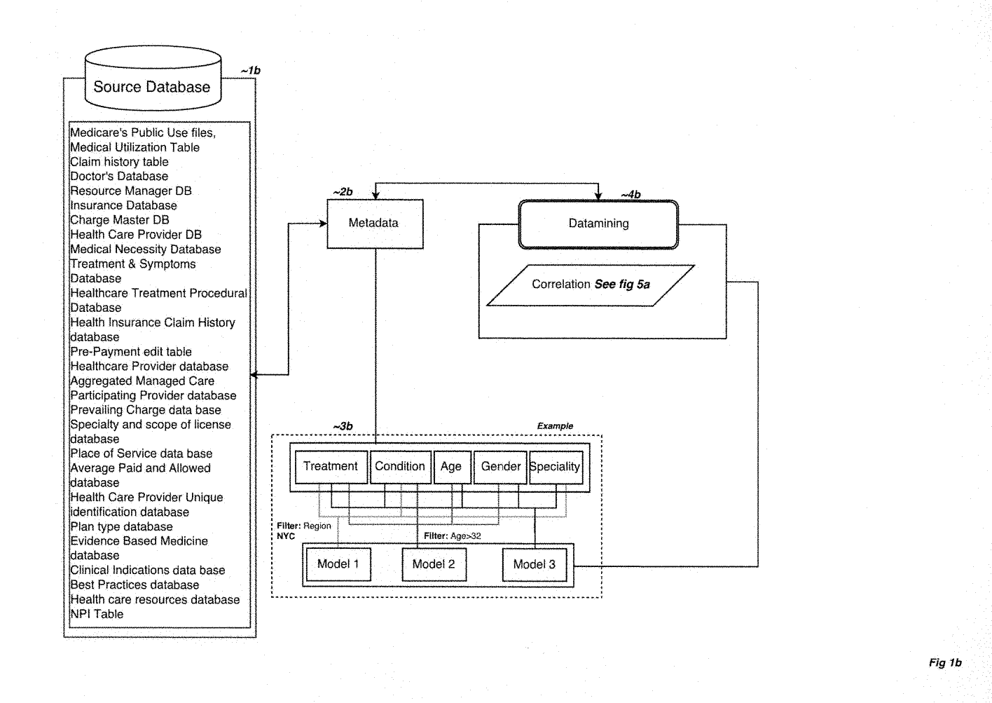

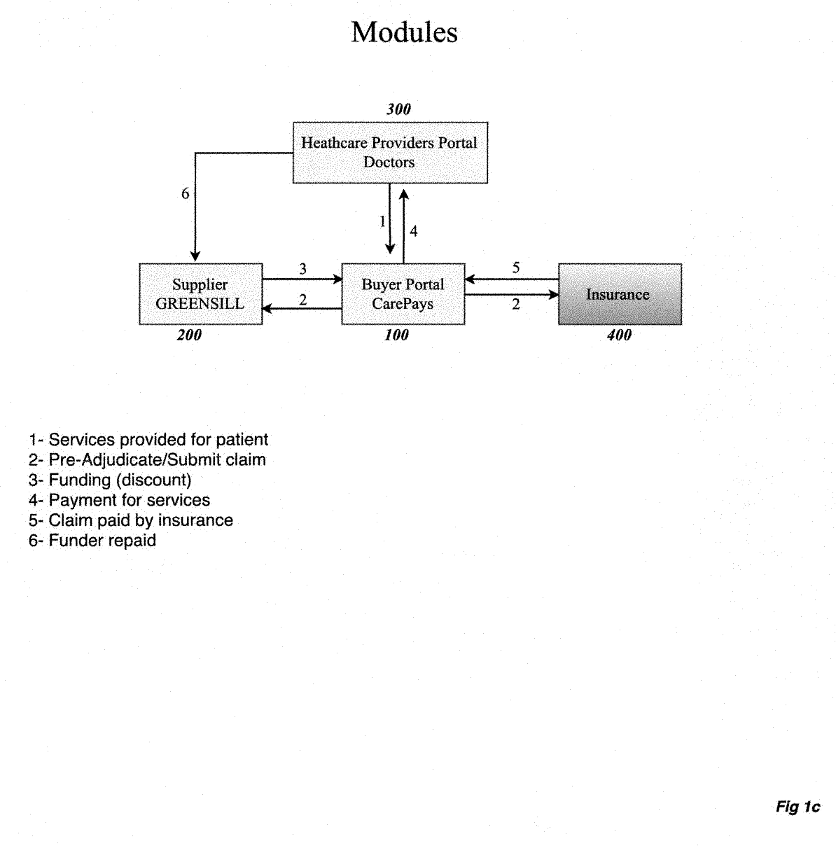

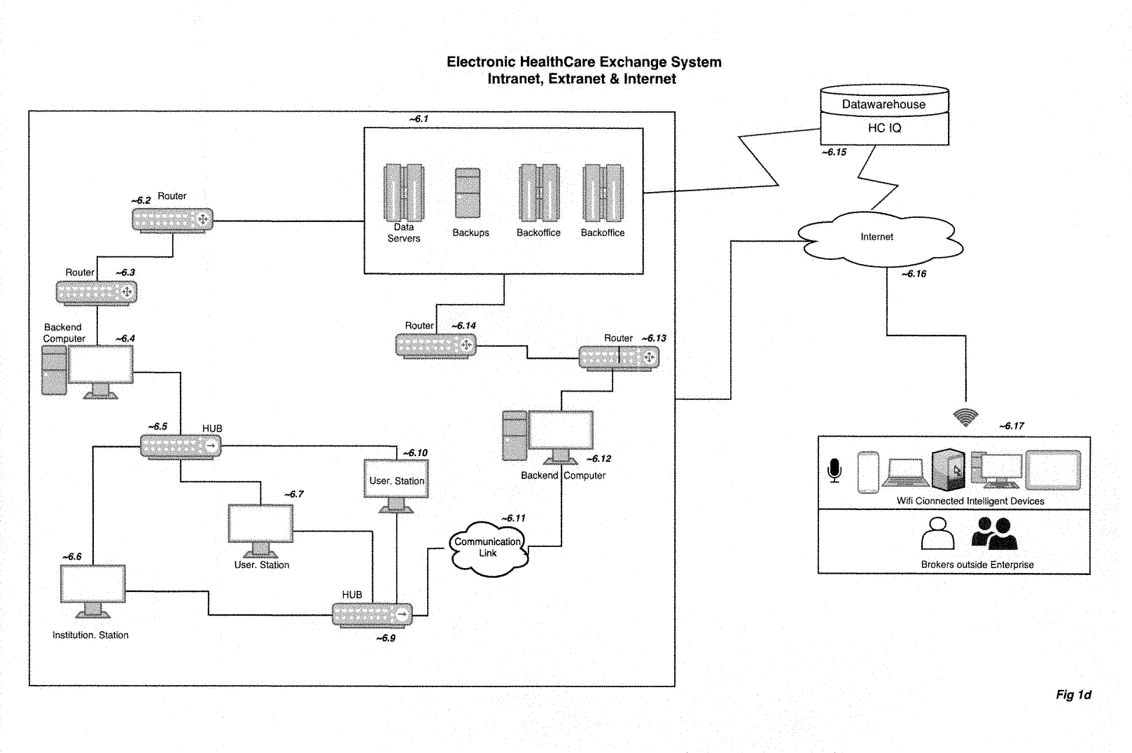

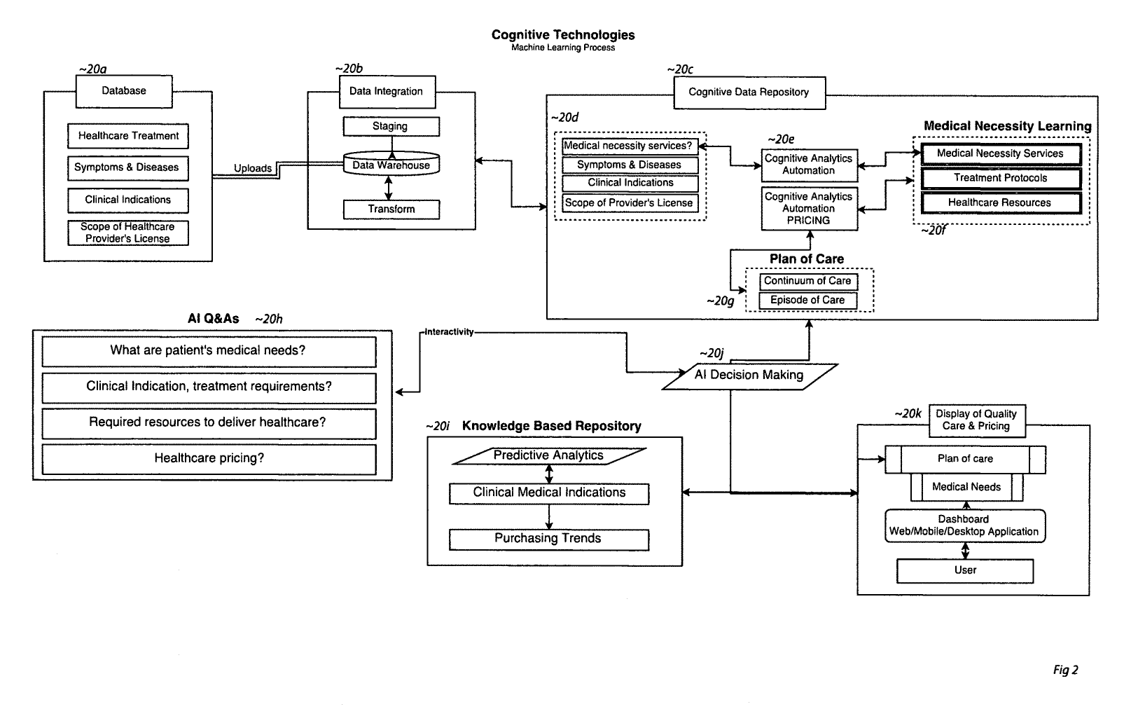

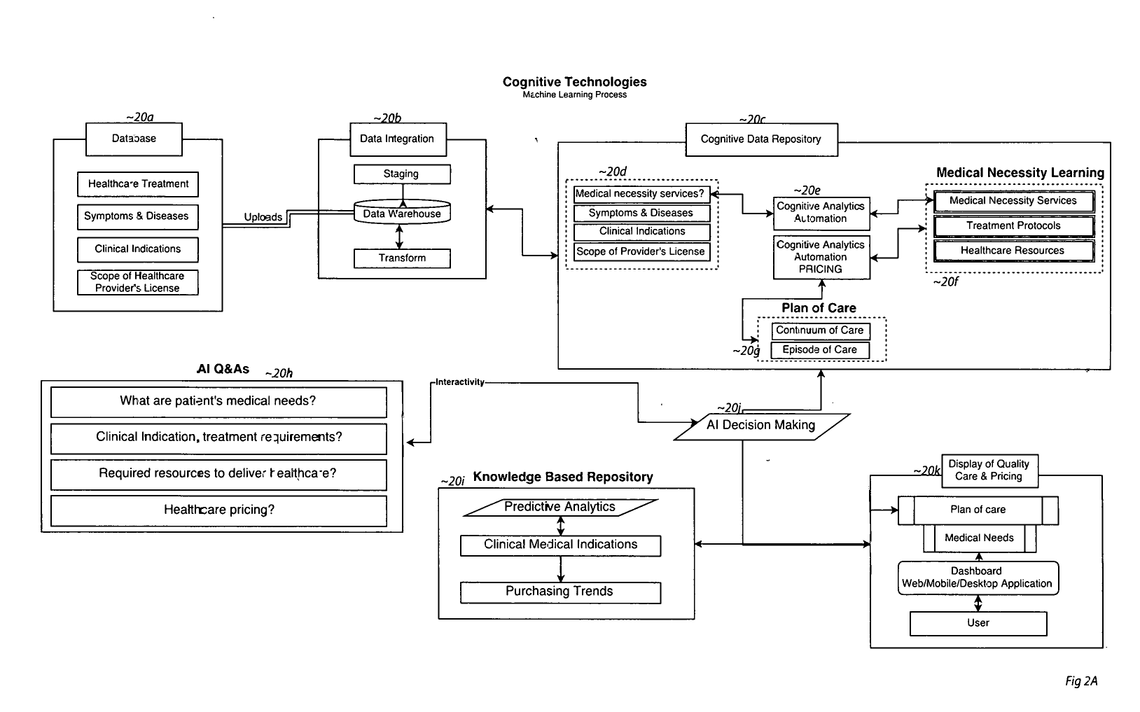

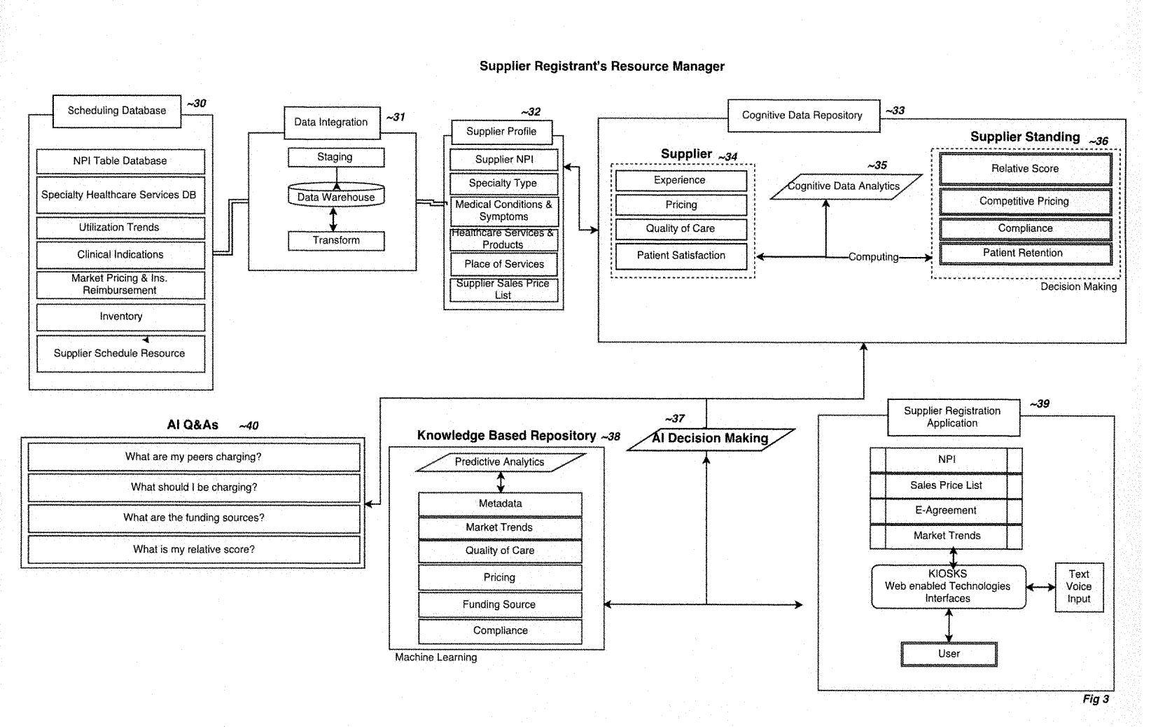

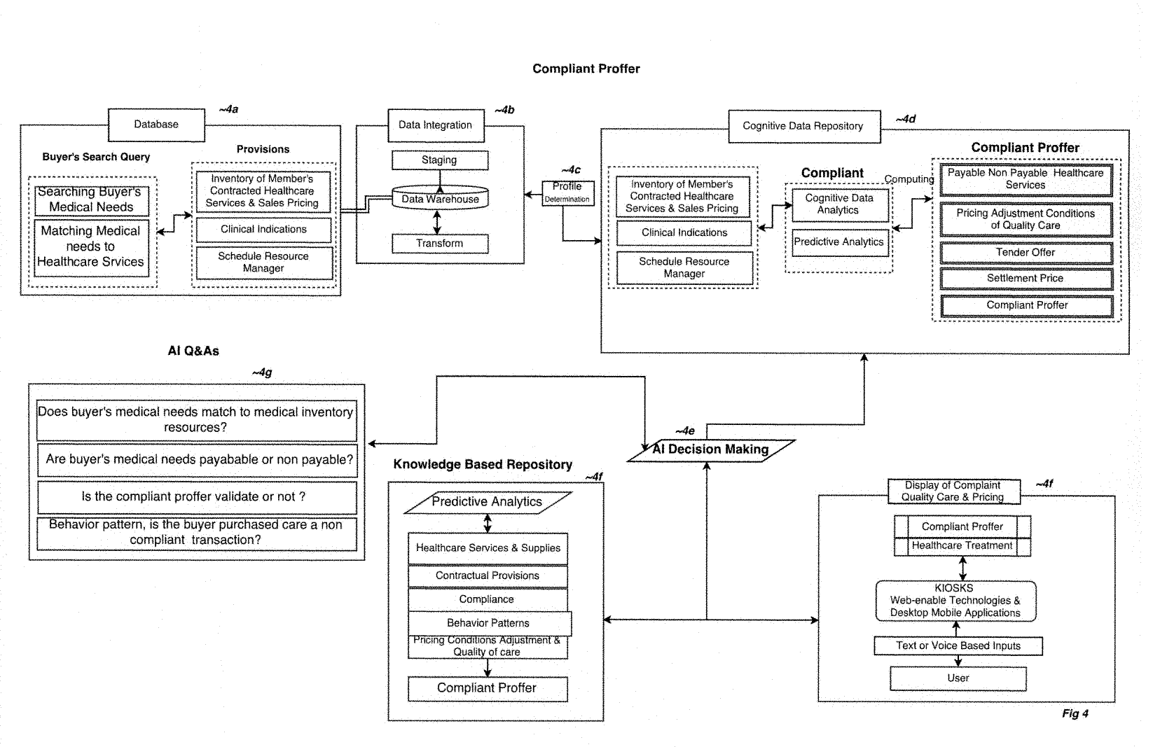

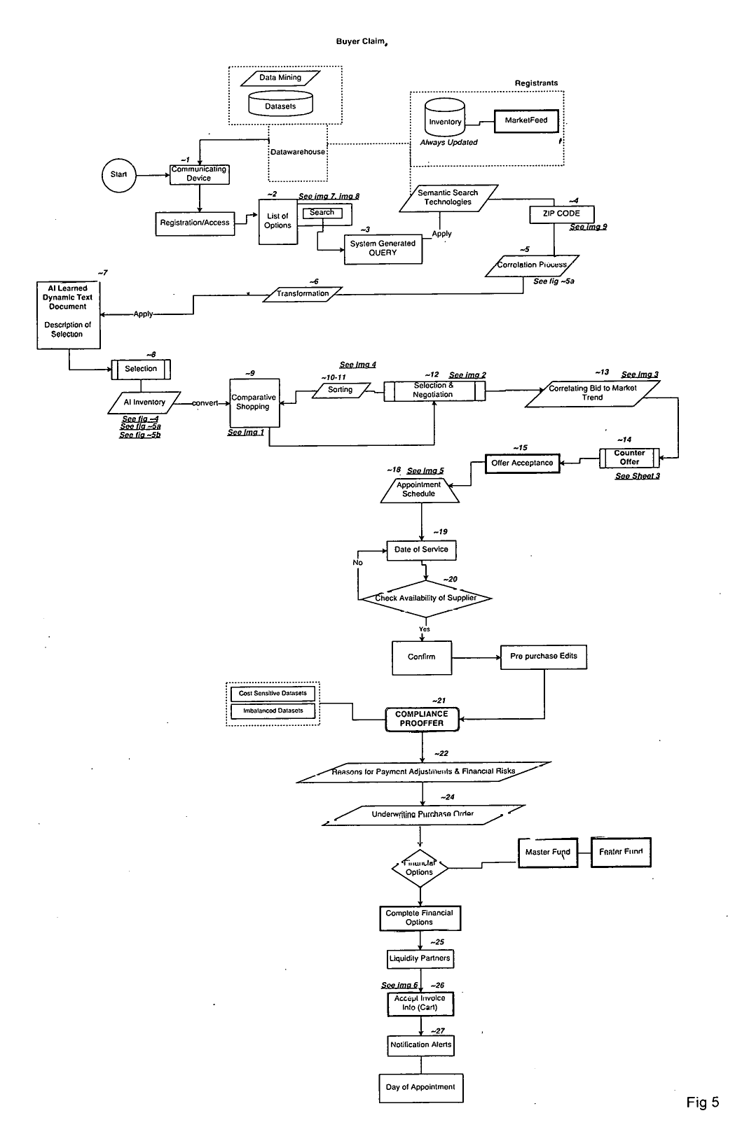

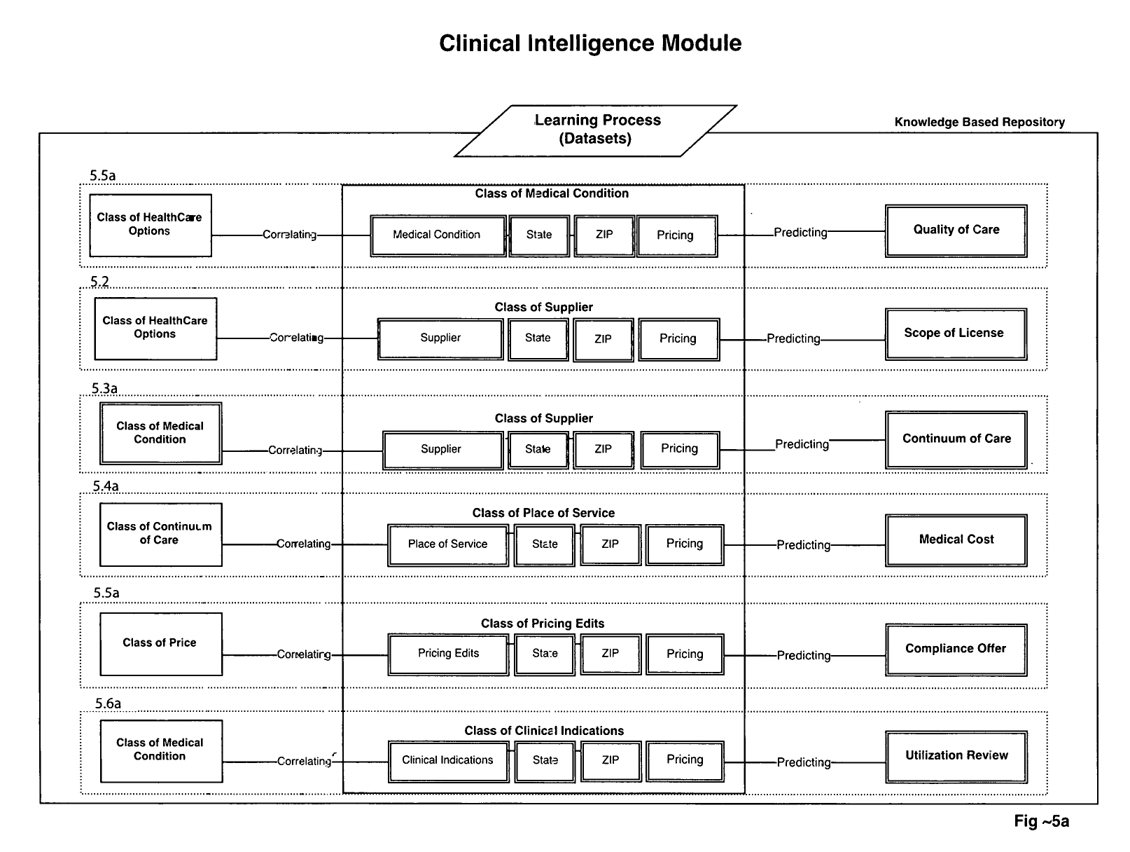

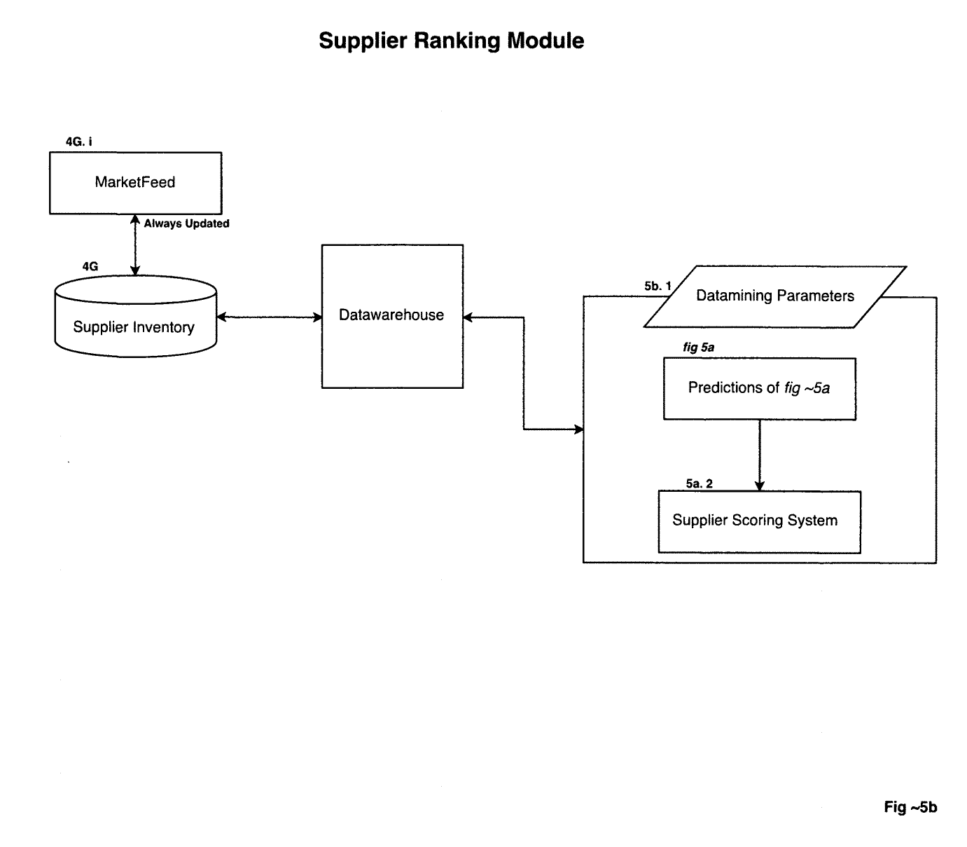

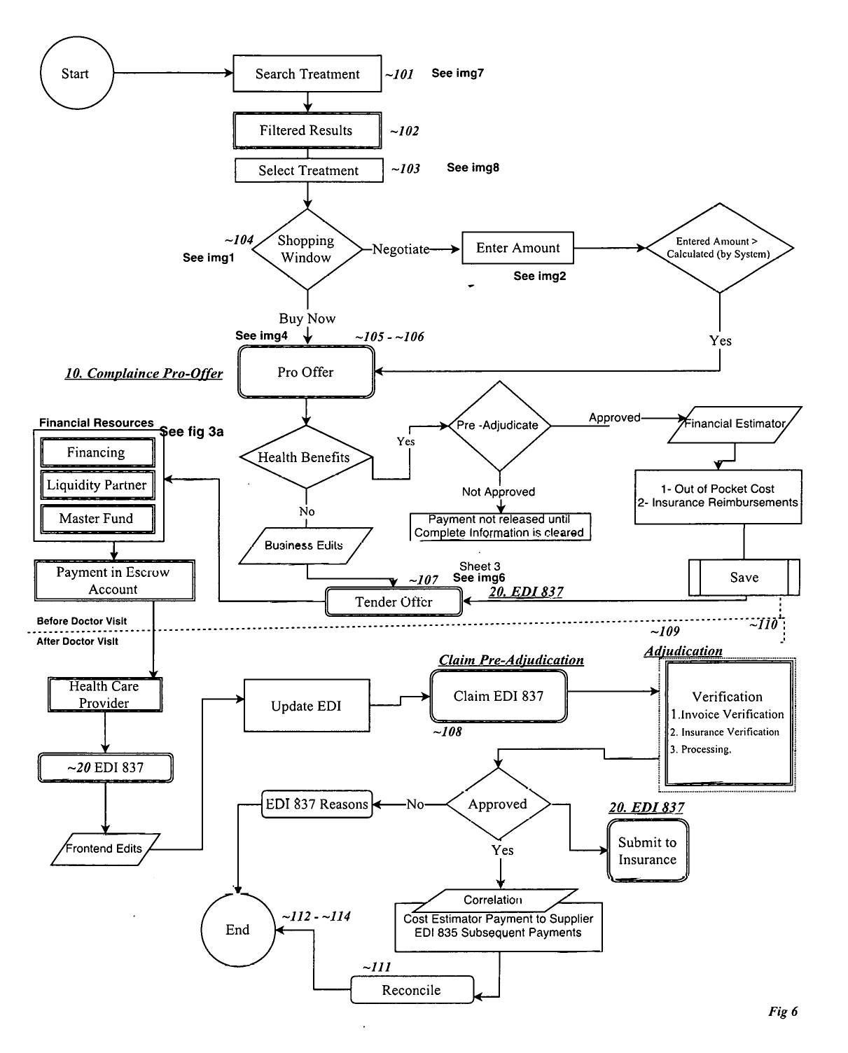

FIG. 2 diagram illustrates a framework that can allow the present invention to easily turn a machine learning algorithm into a solid working application. For example, the present invention could correlate between variables and class labels to build a classifier. The present invention can apply Bayesian theorem for regression and classification problems involved with probability. The automated purchase care exchange system attempts to show the probabilistic relationship between different variables and determine, given the variables, which category it more likely to belongs to.

Example 5 illustrates 8 training examples, two features and one class. Based on input data, a probabilistic relationship was determined between the healthcare option and the diagnosis and/or symptom of the buyer. By observing these relationships, the present invention is capable of establishing a function that more or less mimics this relationship. An intelligent agent is used to conceptualize the environment and detect changes in the environment and reacts to that change based on information from the past observations, and the rules it has been taught 20b.

Association rule learning algorithms were can be established between the itemset and the transactions for these items and items sets. For example, the relationship between X and Y, thus the probability of when you obtain X you can also obtain Y. The compliance rules and conditional pricing instances can be found in the database by observing the itemset and the items therein sheet 1.

The present invention can use clustering algorithms to cluster the available data into groups, sheet 5, where the data points in such a group are more similar to each other than those in other groups.

To decrease the response time, the automated purchase care system may be dedicated or substantially dedicated to performing automated purchasing operations. FIG. 5a diagram illustrates some examples of learning algorithms embedded in FIG. 1 that can be found in an automated purchase care exchange system. For example, Bayesian theorem (Bayesian Algorithms) can be involved with probability for regression and classification problems. In accordance with one aspect of the present invention, the automated purchase care exchange system decides whether or not to submit an order or compliant proffer based on repricing logic and compliant protocols derived from learning algorithms embedded throughout 100-400, sheet 1.

5.1a can be applied to logistic regression. The automated purchase care exchange system establishes a relationship 5.1a-5.6a it seeks to model the relationship between the variables. Over time, rules can be established between the itemset and the transactions for these items and item sets. The relationship between X and Y thus, the probability of when X is obtained you also obtain Y. In this case, when a relationship between the healthcare option and the medical condition are established there is a greater likelihood that the healthcare is medically necessary or predictably quality of care. Conversely, the lower the predictability is the less likely the healthcare is quality of care.

Another example of the present invention of transforming the data into meaningful information. The established relationship between healthcare option and a medical condition can predict the probability that the healthcare option when coupled with a medical condition have a greater likelihood of being purchased together.

In accordance with one embodiment of the present invention, machine learning algorithms can incorporate metadata a10. 100 represents potentially useful metadata

The present invention recognizes that electronic purchase care exchange system computers can produce accessible metadata that could enable the receiving party to have the same ability to access, search, and display . . . information as the producing party; and that metadata is a critical component of any electronically stored data See Sheet 5.

Taken together 100 data sets reflect the variability in metadata availability that is common across healthcare and health insurance industry. The wide array of metadata fields is potentially useful as sources of features for use by a classifier. One class of such data can be stored in transaction databases from which all items obtained in a single transaction can be retrieved as a unit. The transaction can then be electronically examined to determine what items typically appear together, e.g., which healthcare items consumers typically buy together in a database of purchased care transactions. This in turn gives insight into questions such as market healthcare services or products more effectively, how to group them in a display layout (window shopping) or product pages (physical therapy), or which items to offer on sale to boost the sale of other healthcare items.

One objective of the present invention is to focus on determining which groups of healthcare items, called itemsets, frequently appear together in transactions. From any itemset an association rule may be derived which, given the occurrence of a subset of the items in the itemset, predicts the probability of the occurrence of a subset of the remaining items. sheet 5.

What is claimed is:

1-5. (canceled)

6. An electronic client server network system to buy healthcare services and products from the healthcare industry and a processor to sell healthcare to consumers seeking services and/or products, using free and ubiquitous healthcare and health insurance industry metadata, collecting training set and training the model in a standard transfer learning paradigm, processing training data set, applying the collected training set industry protocols to learning algorithms, infusing industry learning experience protocols into financial transactions to ensure compliance with protocol, mitigating excessive charges, unwarranted healthcare, fraud and abuse, and/or adjusting pricing while concurrently scoring risks associated with the transaction to underwrite the transaction to ensure payment for the delivery of healthcare, establishing and operating or maintaining arrangement or contract with healthcare providers (suppliers) relating to (A) the healthcare rendered by the suppliers, and (B) the amounts to be paid to the suppliers for such services, accordingly, producing a compliant proffer to counter a non-compliant bid from an intelligent device, comprises of,
a first computer in electronic communications with a knowledge based electronic purchase care exchange system, comprising of data warehouse techniques, machine learning and data mining of metadata and knowledge base, source data, operational database management systems, with a processor configured with logic for implementing an automated purchase care application and at least one other application through an operating system, wherein the operating system gives priority to the automated purchase care application over the at least one other application,
the first computer (a) receiving electronic communications indicative of bid to purchase healthcare option on the electronic purchase care exchange system (b) automatically conceptualizing specified circumstances that are coupled to training set protocols resident in hardware and software, run with the training dataset producing action, resolving whether the current bid for the healthcare option satisfies the training data set protocols, whether to (1) accept the bid, (2) reject the bid based on learning experiences, or adjust the price based on learning experience (3) transform the bid to a compliant proffer, wherein learning experiences and resolutions are stored in a memory, (c) after using training set, the parameters are subjected to validation ‘test set’; automatically checking conditional pricing and whether one or more knowledge-base-protocol for a healthcare option transaction are satisfied, and (d) automatically transmitting from the electronic purchase care exchange system a response to the current bid within milliseconds of receiving the electronic communications when conditional pricing or one or more knowledge-base-protocols are satisfied, wherein the response includes an order or quote for the healthcare option capable of being matched to the compliant proffer.

7. The system of claim 1, wherein the automated purchasing application is run without debug messages.

8. The system of claim 1, wherein the first computer implements multiple other applications through an operating system, wherein the operating system gives priority to the automated purchasing application over the multiple other applications.

9. The system of claim 1, further comprising the first computer receiving electronic communications from the inventory market feed, and storing the supplier's purchase order data in the memory.

10. The system of claim 4, further comprising the first computer calculating the compliant proffer using at least the stored conditional price data.

11. The system of claim 1, further comprising a computer bus communicatively coupled to the first computer (see a30) the first computer (intelligent device) receiving electronic communications from the automated purchase care exchange system inventory updating market feed, the electronic communications indicative of financial transactions subjected to training data set parameters, deriving supplier purchase order data directly from the electronic communications from the inventory market feed.

12. The system of claim 6, further comprising the computer bus transmitting the qualified suppliers' purchase order based on the buyer's zip and adding the spread to the intelligent device.

13. The system of claim 6, further comprising the computer bus storing the price data in the memory, and the present invention calculating conditional pricing and price targets using at least stored price data.

14. The system of claim 6, further comprising the computer bus transmitting data to the intelligent device and the present invention transmitting comparative display window details including healthcare option price data details.

15. The system of claim 7, further comprising of present invention calculating price target and present invention using at least the inventory supplier purchase order data, and storing price target in memory.

16. The system of claim 1, wherein the operating system is a Windows-based operating system.

17. The system of claim 1, wherein the operating system is a Linux operating system.

18. The system of claim 1, wherein the current price is a current bid price, and the current bid price is subject to train set protocols the current bid price being greater than or equal to the price target of the underlying security of the healthcare option.

19. The system of claim 1, wherein the current proffer is subjected to training data set protocols and the current proffer is a compliant proffer, and the compliant proffer includes the current proffer price being greater than or equal to the price target of the healthcare option.

20. The system of claim 1, wherein training data set protocols, being able to send or receive secured data over one or more communication links of the automated electronic purchase care exchange system.

21. The system of claim 1, wherein training data set protocol include a purchase status of the healthcare option of the underlying security healthcare option.

22. The system of claim 1, wherein training data set protocol include maximum units of the healthcare options.

23. The system of claim 1, wherein training data set protocol include a maximum number of automated purchasing attempts during a predetermined time period.

24. The system of claim 1, wherein training data set protocol include an automated purchasing frequency for the healthcare option.
